(19) United States
(12) Patent Application Publication
(10) Pub. No.: US 2021/0128052 A1
(43) Pub. Date: May 6, 2021

(54) DEVICE FOR MONITORING THE ELECTROPHYSIOLOGICAL ACTIVITY OF A SUBJECT

(71) Applicant: BIOSERENITY, Paris (FR)

(72) Inventors: Pierre-Yves FROUIN, Paris (FR); Pierre PROT, Paris (FR); Marion GOUTHEZ, Paris (FR)

(73) Assignee: BIOSERENITY, Paris (FR)

(21) Appl. No.: 16/478,226

(22) PCT Filed: Jan. 19, 2018
(86) PCT No.: PCT/FR2018/050138

§ 371 Date: Jul. 16, 2019

(30) Foreign Application Priority Data
Jan. 19, 2017 (FR) 1750443

(51) Int. Cl. A61B 5/00; A61B 5/291; A61B 5/296; A61B 5/16; A61B 5/28; A61B 5/1455; A61B 5/01; A61B 5/113

(52) U.S. Cl. CPC A61B 5/4094; A61B 5/291; A61B 5/296; A61B 5/6803; A61B 5/0006; A61B 2562/164; A61B 5/28; A61B 5/14551; A61B 5/01; A61B 5/6805; A61B 5/1135; A61B 5/163

(57) ABSTRACT

A device for monitoring the electro-physiological activity of a subject, particularly for diagnosing or monitoring epilepsy, including:
measurement element for measuring electrical brain activity;
measurement element for measuring the electrical activity of the muscles of mastication;
an acquisition module configured to acquire in synchronously the signals measured by the measurement element for measuring electrical brain activity and by the measurement element for measuring the electrical activity of the muscles of mastication;
   a signal processing modules; and
a transmission module for transmitting the electrical signals acquired by the acquisition module to the signal processing module.

The signal processing module is configured to detect among the signals measured by the measurement element for measuring electrical brain activity the signals measured by the measurement element for measuring the electrical activity of the muscles of mastication.

### FIELD OF THE INVENTION

The present invention relates to a device for monitoring the electro-physiological activity of a subject. In particular, the present invention relates to a device comprising at least one item of clothing suitable for being worn by a subject and enabling the monitoring of the electro-physiological activity of said subject. Said device is particularly configured to enable the diagnosis or monitoring of neurological or physiological diseases or disorders requiring electrical brain activity measurements.

### STATE OF THE RELATED ART

It is known from the prior art, particularly by means of the French patent application FR 3 008 300, a medical device based on the use of an item of smart clothing enabling the processing of a stream of data measured by sensors (EEG, ECG, EMG, etc.) integrated in said item of clothing to monitor a patient's physiological state. The device is equipped with an onboard electronic housing and a battery, and it sends the data automatically to a mobile device or to a remote server. The data stream is analysed by the mobile device or by the remote server which can then trigger various actions such as recording events or alarms if abnormal signals are observed.

However, this device has limitations in bioelectric signal analysis. Indeed, the signals obtained by measuring the electrical brain activity by EEG sensors are very weak. These signals are thus subject to a high risk of interference. This interference can be varied. It may be caused by other electro-physiological activities, such as muscle activity (particularly the cardiac muscle or muscles of mastication), as well as by High-Frequency Hertzian waves and electrostatic or Low-Frequency type interference. The electrical mains voltage is also liable to generate noise at 50 Hz or 60 Hz on the signals measured.

This interference impedes the processing and interpretation of the signals, whether interpretation is performed manually by technicians or medical practitioners; or electronically by algorithms. This interference may go as far as rendering the characterisation of an alarm impossible, falsifying a diagnosis or issuing same with reservations.

It is known from the prior art the article by Gizel et al. (“Interference of tonic muscle activity on the EEG: a single motor unit study”, Frontiers in human neuroscience, 2014, vol. 8) describing a device comprising an electroencephalogram, for the acquisition of signals produced by a subject's brain activity, and intramuscular electrodes, for the acquisition of signals generated by the activity of the muscles of mastication. Gizel et al. describes the use of the signals acquired by the intramuscular electrodes to detect artefacts associated with the activity of the muscles of mastication in EEG signals. However, this device has the limitation of using invasive electrodes that must be inserted in the muscles of mastication.

There is also known from the prior art the U.S. patent application US 2011/257517 describing a system for detecting the onset of an attack specific to a subject. Said system comprises a means for the acquisition of the electrical brain activity and a means for measuring the electrical activity of the muscles of mastication. However, this system is not suitable for detecting interference in the electrical brain signals which are caused by the electrical activity of the muscles of mastication.

It is known from the prior art the U.S. patent application US 2012/203079 describing a system for the acquisition of a subject's electrical brain activity. Said system comprises electrodes for detecting the electrical brain activity and electrodes for the acquisition of artefacts. However, the electrodes described by US 2012/203079 are implanted in the subject's body, the use of such a system therefore involves an invasive step of implanting said electrodes.

It is also known from the prior art the U.S. patent application US 2012/203133 describing a system comprising a means for the acquisition of a subject's electrical brain and muscle activity. However, said system has the limitation of not incorporating a module for detecting artefacts associated with the muscle activity present in the electrical brain signals.

The aim of the present invention is therefore that of providing a device for monitoring the electro-physiological activity of a subject suitable for remedying the major drawbacks of the prior art. In particular, the present invention enables a reliable analysis of the signals measured by detecting the periods during which the signals are disturbed and by enabling coupling of multiple bio-signals for superior interpretation of the markers in the patient's condition.

### SUMMARY

The present invention relates to a device for monitoring the electro-physiological activity of a subject, particularly for diagnosing or monitoring epilepsy, comprising:
a means for measuring electrical brain activity;
a means for measuring the electrical activity of the muscles of mastication; the means for measuring the electrical activity of the muscles of mastication including at least one surface electrode integrated in a textile element, said textile element including a means for holding said at least one electrode in contact with a portion of a subject's head;
an acquisition means configured to acquire in synchronously the signals measured by the means for measuring electrical brain activity and by the means for measuring the electrical activity of the muscles of mastication;
a signal processing means; and
a means for transmitting the electrical signals acquired by the acquisition means to said signal processing means;
   wherein said signal processing means is configured to detect among the signals measured by the means for measuring electrical brain activity the signals measured by the means for measuring the electrical activity of the muscles of mastication.

According to one embodiment, the device further comprises a means for measuring the electrical activity of the deltoid muscles or the trapezius muscles, wherein the acquisition means is also configured to acquire synchronously the signals measured by the means for measuring the electrical activity of the deltoid muscles or the trapezius muscles, and the signal processing means is also configured to detect among the signals measured by the means for measuring the electrical activity of the deltoid muscles or the trapezius muscles signals correlated with predefined movements.

According to one embodiment, the device further comprises a means for measuring eye movement, wherein the acquisition means is also configured to acquire synchronously the signals measured by the means for measuring eye movement, and the signal processing means is also configured to detect among the signals measured by the means for measuring electrical brain activity the signals measured by the means for measuring eye movement.

According to one embodiment, the device further comprises a means for measuring electrical cardiac muscle activity, wherein the acquisition means is also configured to acquire synchronously the signals measured by the means for measuring electrical cardiac muscle activity, and the signal processing means is also configured to detect among the signals measured by the means for measuring electrical cardiac muscle activity heartbeats.

According to one embodiment, the device further comprises a means for measuring respiratory muscle activity, wherein the acquisition means is also configured to acquire synchronously the signals measured by the means for measuring respiratory muscle activity, and the signal processing means is also configured to detect among the signals measured by the means for measuring electrical cardiac muscle activity parasitic signals correlated with the signals measured by the means for measuring respiratory muscle activity.

According to one embodiment, the device further comprises a means for measuring external electromagnetic interference, wherein the acquisition means is also configured to acquire synchronously the signals measured by the means for measuring external electromagnetic interference, and the signal processing means is also configured to detect among the signals measured by the means for measuring electrical brain activity the signals measured by the means for measuring external electromagnetic interference.

According to one embodiment, the device further comprises a means for measuring movement, wherein the acquisition means is also configured to acquire synchronously the signals measured by the means for measuring movement, and the signal processing means is also configured to detect among the signals measured by the means for measuring electrical cardiac muscle activity parasitic signals correlated with the signals measured by the means for measuring movement.

According to one embodiment, the device further comprises a means for measuring the blood oxygen concentration and/or a means for measuring electromagnetic radiation, particularly visible electromagnetic radiation, and/or a means for measuring body temperature.

According to one embodiment, the device comprises a cap type item of clothing suitable for being worn on the subject's head and at the level of the subject's muscles of mastication; said cap type item of clothing comprising the means for measuring electrical brain activity and the means for measuring the electrical activity of the muscles of mastication; said means for measuring electrical brain activity comprising a plurality of conductive electrodes, and said means for measuring the electrical activity of the muscles of mastication comprising at least one conductive electrode.

According to one embodiment, the device comprises a vest type item of clothing suitable for being worn on the subject's chest; said vest type item of clothing comprising the means for measuring the electrical activity of the deltoid muscles or trapezius muscles, the means for measuring electrical cardiac muscle activity and/or the means for measuring respiratory muscle activity; said means for measuring the electrical activity of the deltoid muscles or trapezius muscles comprising a plurality of conductive electrodes; said means for measuring cardiac muscle activity comprising at least one conductive electrode; and said means for measuring respiratory muscle activity comprising a textile elastic strip preferably comprising a capacitive means for measuring the rib-cage spread.

According to one embodiment, the cap type item of clothing or the vest type item of clothing are textile items of clothing, and the conductive electrodes comprise textile electrodes woven, knit, stitched, deposited or printed in or on said items of textile clothing.

According to one embodiment, said signal processing means comprises a memory unit, said memory unit comprising neurological markers; and the signal processing means is configured to identify a neurological disorder among the signals measured by the means for measuring electrical brain activity based on the neurological markers.

### Definitions

In the present invention, the terms hereinafter are defined as follows:
“At the level of” denotes at the height of. Thus, by way of example, an item of clothing suitable for being worn at the level of a mastoid process is an item of clothing suitable for covering the overlying skin of the mastoid process.
“Textile electrodes” denotes electrodes consisting of conductive wires or of wires covered with a conductive material woven, knit, stitched, deposited or printed in or the item of clothing. According to one embodiment, the wire is a metallic wire or a wire covered with a metallic coating, in particular, the wire may be a silver wire or a silver-coated wire. According to one embodiment, the wire is a carbon wire or a wire comprising conductive particles such as metallic nanotubes or nanoparticles. According to one embodiment, the wire is covered with an intrinsically conductive material, such as polyaniline.
“Muscles of mastication” denotes the muscles enabling the movement of the mandible during mastication and thus the rising and lowering of the jaw.
“Cap type item of clothing” denotes an item of textile clothing worn next the skin, if applicable in contact with hair such as head hair, covering the subject's scalp, it particularly includes caps.
“Vest type item of clothing” denotes an item of textile clothing worn next the skin, if applicable in contact with hair, covering the subject's chest, it particularly includes vests, tee-shirts or tank tops, that equally well may be long-sleeved, short-sleeved or sleeveless.
“Signal processing means” denotes at least one microprocessor, at least one integrated circuit, at least one electronic board and at least one microcontroller. This signal processing means may comprise an assembly comprising including an onboard computer and external computing means such as a mobile device and remote servers.

### DETAILED DESCRIPTION

The present invention present invention relates to a device for monitoring the electro-physiological activity of a subject particularly for diagnosing or monitoring neurological or physiological diseases or disorders requiring electrical brain activity measurements, in particular for diagnosing or monitoring epilepsy.

The device according to the present invention comprises a means for measuring electrical brain activity, a means for measuring the electrical activity of the muscles of mastication, an acquisition means, a transmission means and a signal processing means.

The acquisition means is configured to acquire synchronously the signals measured by the means for measuring electrical brain activity and by the means for measuring the electrical activity of the muscles of mastication.

A synchronised acquisition of these signals enables a comparison on the same date or at the same time t.

The transmission means is configured to transmit the electrical signals acquired by the acquisition means to the signal processing means. In one embodiment, the signal processing means is remote from this device and is connected wirelessly. In one embodiment, the signal processing means is remote from this device and is connected in a wired manner.

In one embodiment, the device comprises a plurality of acquisition means, transmission means and signal processing means.

The signal processing means is configured to detect among the signals measured by the means for measuring electrical brain activity the signals measured by the means for measuring the electrical activity of the muscles of mastication. These signals may subsequently be attenuated in the electrical brain signal or highlight such that the interpreter identifies them clearly as such. This detection uses a breakdown into individual components and into principal components (ICA and PCA) or indeed correlation analyses known to those skilled in the art.

In view of the low intensity of electrical brain activity signals, the signals measured are frequently “contaminated” with parasitic signals, and in particular, the electrical signals emitted by the muscles of mastication, due to the intensity thereof and the proximity thereof with the brain. It is therefore necessary to differentiate electrical brain activity signals from the other parasitic signals so as to enable a reliable analysis of the electrical brain activity signals.

According to one embodiment, the device according to the invention also comprises a means for measuring electrical muscle activity. This embodiment makes it possible particularly to detect repetitive muscle movements occurring during certain epileptic seizures. These movements may be “clonic” type movements or severe and sudden muscle contractions with or without associated movement. The muscle symptoms thus observed particularly enable medical personnel to identify the type of epilepsy: focal epilepsy or generalised epilepsy for example. In this embodiment, the acquisition means is also configured to acquire synchronously the signals measured by the means for measuring electrical muscle activity, and the signal processing means is also configured to detect among the signals measured by the means for measuring electrical muscle activity signals correlated with predefined movements; in particular sudden, jerky or repetitive movements. This detection may be carried out using an amplitude demodulation technique known to those skilled in the art.

According to one embodiment, the device according to the invention also comprises a means for measuring the electrical activity of the deltoid muscles or the trapezius muscles. This embodiment makes it possible particularly to detect repetitive muscle movements occurring during certain epileptic seizures. These movements may be “clonic” type movements or severe and sudden muscle contractions with or without associated movement. The muscle symptoms thus observed particularly enable medical personnel to identify the type of epilepsy: focal epilepsy or generalised epilepsy for example. In this embodiment, the acquisition means is also configured to acquire synchronously the signals measured by the means for measuring the electrical activity of the deltoid muscles or the trapezius muscles, and the signal processing means is also configured to detect among the signals measured by the means for measuring the electrical activity of the deltoid muscles or the trapezius muscles signals correlated with predefined movements; in particular sudden, jerky or repetitive movements. This detection may be carried out using an amplitude demodulation technique known to those skilled in the art.

According to one embodiment, the device according to the invention also comprises a means for measuring eye movement (or electro-oculogram). This embodiment makes it possible particularly to identify parasitic signals due to eye movements among the signals measured by the means for measuring electrical brain activity. In one embodiment, the acquisition means is also configured to acquire synchronously the signals measured by the means for measuring eye movement, and the signal processing means is also configured to detect among the signals measured by the means for measuring electrical brain activity the signals measured by the means for measuring eye movement. This detection uses a breakdown into individual components and into principal components (ICA and PCA) or indeed correlation analyses known to those skilled in the art.

According to one embodiment, the device according to the invention also comprises a means for measuring electrical cardiac muscle activity. In this embodiment, the acquisition means is also configured to acquire synchronously the signals measured by the means for measuring electrical cardiac muscle activity, and the signal processing means is also configured to detect among the signals measured by the means for measuring electrical cardiac muscle activity heartbeats or heartbeat rate variations. This detection uses a QRS peak detection algorithm known to those skilled in the art among which mention may be made by way of example of the El-Gendi algorithm or the Pan-Thompkins algorithm. According to one embodiment, the signal processing means is also configured to identify anomalies due to cardiac activity in electrical brain activity signals. This detection uses a breakdown into individual components and into principal components (ICA and PCA) or indeed correlation analyses known to those skilled in the art.

According to one embodiment, the device according to the invention also comprises a means for measuring respiratory muscle activity. In this embodiment, the acquisition means is also configured to acquire synchronously the signals measured by the means for measuring respiratory muscle activity.

According to one embodiment, the device according to the invention also comprises a means for measuring electrical cardiac muscle activity and a means for measuring respiratory muscle activity. In this embodiment, the acquisition means is also configured to acquire synchronously the signals measured by the means for measuring electrical cardiac muscle activity and the signals measured by the means for measuring respiratory muscle activity, and the signal processing means is also configured to detect among the signals measured by the means for measuring electrical cardiac muscle activity parasitic signals correlated with the signals measured by the means for measuring respiratory muscle activity. This detection uses a breakdown into individual components and into principal components (ICA and PCA) or indeed correlation analyses known to those skilled in the art. In one embodiment, the signal processing means is configured to identify the respiratory activity based on the signals measured by the means for measuring electrical cardiac muscle activity. Indeed, the heart is subject to mechanical stress during respiration, for this reason, the interval between beats changes as a function of respiration and the amplitude of the beats changes during respiration. Thus, it is possible to estimate the respiration according to the signals measured by the means for measuring electrical cardiac muscle activity.

According to one embodiment, the device according to the invention also comprises a means for measuring external electromagnetic interference. In this embodiment, the acquisition means is also configured to acquire synchronously the signals measured by the means for measuring external electromagnetic interference, and the signal processing means is also configured to detect among the signals measured by the means for measuring electrical brain activity the signals measured by the means for measuring external electromagnetic interference. This detection uses a breakdown into individual components and into principal components (ICA and PCA) or indeed correlation analyses known to those skilled in the art. According to one embodiment, the signal processing means is also configured to remove from the signals measured by means for measuring electrical brain activity, the signals measured by the means for measuring electromagnetic interference. This removal may be carried out using a breakdown into individual components and into principal components (ICA and PCA).

According to one embodiment, the device according to the invention also comprises a means for measuring movement. In this embodiment, the acquisition means is also configured to acquire synchronously the signals measured by the means for measuring movement. According to one embodiment, the means for measuring movement is an accelerometer. According to one embodiment, the signal processing means is configured to detect physical exertion based on the signals measured by the means for measuring movement. According to one embodiment, the signal processing means is configured to compute the subject's energy expenditure based on the signals measured by the means for measuring movement. According to one embodiment, the signal processing means is configured to identify the subject's movements and posture based on the signals measured by the means for measuring movement. These operations are carried out using a 3-axis, 6-axis or 9-axis accelerometer. These signals are then analysed according to signal processing (filtering, integration, thresholding) and classification methods with respect to models. This analysis is known to those skilled in the art, and results in a classification of the subject's posture, energy expenditure (knowing the subject's mass), and an estimation of the subject's energy expenditure.

According to one embodiment, the device according to the invention also comprises a means for measuring electrical cardiac muscle activity and a means for measuring movement. In this embodiment, the means for measuring movement is configured to measure the subject's movement, and the acquisition means is also configured to acquire synchronously the signals measured by the means for measuring electrical cardiac muscle activity and the signals measured by the means for measuring movement. According to one embodiment, the means for measuring movement is an accelerometer. According to one embodiment, the signal processing means is also configured to detect among the signals measured by the means for measuring electrical cardiac muscle activity parasitic signals correlated with the signals measured by the means for measuring movement. This detection uses a breakdown into individual components and into principal components (ICA and PCA) or indeed correlation analyses known to those skilled in the art.

According to one embodiment, the device according to the invention also comprises a means for measuring electromagnetic radiation, preferably a means for measuring electromagnetic radiation in the visible range. In this embodiment, the acquisition means is also configured to acquire synchronously the signals measured by the means for measuring electromagnetic radiation. According to one embodiment, the means for measuring electromagnetic radiation is a photosensitive sensor.

According to one embodiment, the device according to the invention also comprises at least one means for measuring the blood oxygen concentration. In this embodiment, the acquisition means is also configured to acquire synchronously the signals measured by the means for measuring the blood oxygen concentration. According to one embodiment, the means for measuring the blood oxygen concentration is an optical sensor in contact with the skin.

According to one embodiment, the device according to the invention also comprises a means for measuring body temperature. In this embodiment, the acquisition means is also configured to acquire synchronously the signals measured by the means for measuring body temperature.

According to one embodiment, the device according to the invention also comprises an external video recording means, such as a camera: either an external camera coupled with the device, or a camera integrated in a smartphone, tablet or computer type device. In this embodiment, the acquisition means is also configured to acquire synchronously the signals measured by external video recording means.

According to one embodiment, the signal processing means comprises a memory unit, said memory unit comprising neurological, particularly epileptic, markers; and the signal processing means is configured to identify a neurological, particularly epileptic, activity among the signals measured by the means for measuring electrical brain activity based on the neurological, particularly epileptic, markers. This identification used an epileptic activity recognition algorithm based on a neural network analysis.

According to one embodiment, the device according to the invention also comprises at least one remote storage means and means for communicating to said at least one cloud computing or remote server type remote storage means.

According to one embodiment, the signal processing means is configured to identify or annotate the side-effects of a medical treatment via an electronic data collection. This collection may be direct (input of subject's data in a software application) or indirect (collection via a third-party computing system). In particular, the side-effects resulting in changes to electrical cardiac muscle activity, electrical brain activity, the electrical activity of the muscles of mastication, the electrical activity of the deltoid muscles or trapezius muscles, respiratory muscle activity, electrical muscle activity, blood oxygen concentration and/or body temperature.

According to one embodiment, the signal processing means is configured to identify the subject's sleep cycles based on the various signals acquired by the acquisition means.

In one embodiment as illustrated in FIG. 1, the device according to the invention is a cap type item of clothing 1 suitable for being worn on the subject's head and at the level of the subject's muscles of mastication. According to one preferred embodiment, the device according to the invention is a cap type item of clothing 1 suitable for being worn on the subject's head, at the level of the muscles of mastication and at the level of at least one mastoid process of the subject. Said cap type item of clothing 1 comprises the means for measuring electrical brain activity and the means for measuring the electrical activity of the muscles of mastication.

In one embodiment, the cap type item of clothing 1 suitable for being worn on the subject's head and at the level of the muscles of mastication of the subject 1 comprises a plurality of conductive electrodes suitable for measuring electrical brain activity 1.1 and at least one conductive electrode suitable for measuring the electrical activity of the muscles of mastication 1.2. According to one embodiment, said conductive electrodes are conductive electrodes not requiring gel for use, preferentially said conductive electrodes are dry conductive electrodes. According to one embodiment, the plurality of conductive electrodes suitable for measuring electrical brain activity 1.1 are dry conductive electrodes comprising an ion gel. According to one embodiment, the means for measuring electrical brain activity is a plurality of capacitive electrodes.

According to one embodiment, the plurality of conductive electrodes suitable for measuring electrical brain activity 1.1 is placed according to the 10-20 international system. Alternatively, the plurality of conductive electrodes suitable for measuring electrical brain activity 1.1 may be placed according to any other system, such as the 10-10 system or the 10-5 system.

According to one embodiment, the cap type item of clothing 1 suitable for being worn on the subject's head and at the level of the subject's muscles of mastication comprises 21 conductive electrodes suitable for measuring electrical brain activity 1.1; said conductive electrodes being preferably positioned according to the 10-20 international system.

According to one alternative embodiment, the cap type item of clothing 1 suitable for being worn on the subject's head and at the level of the subject's muscles of mastication comprises a plurality of slots suitable for receiving a plurality of conductive electrodes suitable for measuring electrical brain activity 1.1.

According to one embodiment, the at least one conductive electrode suitable for measuring the electrical activity of the muscles of mastication 1.2 is located on the cap type item of clothing 1 so as to be positioned at the level of the mandible when the cap type item of clothing 1 is worn by the subject. According to this embodiment, the cap type item of clothing 1 comprises at least one textile portion suitable for being positioned at the level of the muscles of mastication when the cap type item of clothing 1 is worn by the subject. In this embodiment, the at least one conductive electrode suitable for measuring the electrical activity of the muscles of mastication 1.2 is a textile electrode situated in said at least one textile portion.

According to one embodiment, the cap type item of clothing 1 suitable for being worn on the subject's head and at the level of the subject's muscles of mastication comprises two conductive electrodes suitable for measuring the electrical activity of the muscles of mastication 1.2 located on the cap type item of clothing 1 so as to be positioned at the level of the mandible when the cap type item of clothing 1 is worn by the subject.

According to one embodiment, the cap type item of clothing 1 comprises at least two textile portions suitable for being positioned at the level of the mandible on either side of the subject's head when the cap type item of clothing 1 is worn by the subject. In one embodiment, the two conductive electrodes suitable for measuring the electrical activity of the muscles of mastication 1.2 are two textile electrodes, each being situated in one of the two textile portions. In one embodiment, the two conductive electrodes suitable for measuring the electrical activity of the muscles of mastication 1.2 are two textile electrodes situated in the same textile portion. According to one embodiment, said conductive electrodes are conductive electrodes not requiring gel for use, preferentially said conductive electrodes are dry conductive electrodes.

According to one embodiment, the conductive electrodes suitable for measuring the electrical activity of the muscles of mastication 1.2 are textile electrodes.

According to one embodiment, the cap type item of clothing 1 suitable for being worn on the subject's head and at the level of the subject's muscles of mastication also comprises at least one reference electrode 1.3 serving as a reference for the electrodes of the cap type item of clothing 1 so as to measure a difference in electric potential. According to one embodiment, this at least one reference electrode 1.3 is located on the cap type item of clothing 1 so as to be positioned behind one of the subject's ears when the cap type item of clothing 1 is worn by the subject. According to one embodiment, this at least one reference electrode 1.3 is located on the cap type item of clothing 1 so as to be positioned at the level of the subject's mastoid process when the cap type item of clothing 1 is worn by the subject.

According to one embodiment, said at least one reference electrode does not require gel for use, preferentially said at least one reference electrode is a dry conductive electrode. According to one embodiment, the at least one reference electrode is a textile electrode.

According to one embodiment, the cap type item of clothing 1 suitable for being worn on the subject's head and at the level of the subject's muscles of mastication also comprises a means for measuring electrical cardiac muscle activity. In one embodiment, the measurement may be carried out by at least one conductive electrode. In one embodiment, the measurement may be carried out by photoplethysmography using an optical device such as an oximeter.

According to one embodiment, the cap type item of clothing 1 suitable for being worn on the subject's head and at the level of the subject's muscles of mastication also comprises a means for measuring the blood oxygen concentration. According to one embodiment, the means for measuring the blood oxygen concentration is an oximeter located on the cap type item of clothing 1 so as to be positioned in or on an ear or on the skin at the level of the subject's neck when the cap type item of clothing 1 is worn by the subject.

According to one embodiment, the cap type item of clothing 1 suitable for being worn on the subject's head and at the level of the subject's muscles of mastication also comprises a means for measuring electromagnetic radiation.

According to one embodiment, the cap type item of clothing 1 comprises a plurality of conductive electrodes suitable for measuring eye movement.

According to one embodiment, the cap type item of clothing 1 suitable for being worn on the subject's head and at the level of the subject's muscles of mastication also comprises a means for measuring movement.

According to one embodiment, the cap type item of clothing 1 suitable for being worn on the subject's head and at the level of the subject's muscles of mastication also comprises a means for measuring external electromagnetic interference.

According to one embodiment, the cap type item of clothing 1 suitable for being worn on the subject's head and at the level of the subject's muscles of mastication also comprises a means for measuring the subject's body temperature.

According to one embodiment, the cap type item of clothing 1 also comprises a space suitable for receiving an electronic system 1.5 comprising the electrical signal acquisition means, signal processing means and transmission means. According to one embodiment the cap type item of clothing 1 also comprises a space suitable for receiving a battery 1.4 enabling the electrical power supply of the electronic system.

In one embodiment as illustrated in FIG. 2A and FIG. 2B, the device according to the invention also comprises a vest type item of clothing 2 suitable for being worn on the subject's chest. Said vest type item of clothing 2 comprises the means for measuring the electrical activity of the deltoid muscles or the trapezius muscles, the means for measuring electrical cardiac muscle activity and/or the means for measuring respiratory muscle activity.

According to one embodiment, the vest type item of clothing 2 comprises a plurality of conductive electrodes suitable for measuring the electrical activity of the deltoid muscles 2.1 or of the trapezius muscles 2.2. According to one embodiment, the means for measuring the electrical activity of the deltoid muscles 2.1 or of the trapezius muscles 2.2 comprises a plurality of conductive electrodes not requiring gel for use. According to one embodiment, the means for measuring the electrical activity of the deltoid muscles or of the trapezius muscles comprises a plurality of dry conductive electrodes. According to one embodiment, the vest type item of clothing 2 comprises 2, 3, 4, 6, 8, 10, 12 or more conductive electrodes, preferentially 4, suitable for measuring the electrical activity of the deltoid muscles 2.1. According to one preferential embodiment, the vest type item of clothing 2 comprises 2 conductive electrodes suitable for measuring the electrical activity of the right deltoid muscles and 2 conductive electrodes suitable for measuring the electrical activity of the left deltoid muscles. According to one embodiment, the vest type item of clothing 2 comprises 2, 3, 4, 6, 8, 10, 12 or more conductive electrodes, preferentially 4, suitable for measuring the electrical activity of the trapezius muscles 2.2. According to one preferential embodiment, the vest type item of clothing 2 comprises 2 conductive electrodes suitable for measuring the electrical activity of the right trapezius muscles and 2 conductive electrodes suitable for measuring the electrical activity of the left trapezius muscles.

According to one embodiment, the vest type item of clothing 2 is suitable for being worn at the level of the deltoid muscles and/or the trapezius muscles and comprises a plurality of conductive electrodes conductrices located on the vest type item of clothing 2 so as to be positioned at the level of the deltoid muscles or of the trapezius muscles when the vest type item of clothing 2 is worn by the subject.

According to one embodiment, the conductive electrodes suitable for measuring the electrical activity of the deltoid muscles 2.1 or of the trapezius muscles 2.2 are textile electrodes.

According to one embodiment, the vest type item of clothing 2 comprises a plurality of electrodes suitable for measuring electrical cardiac muscle activity 2.3. According to one embodiment, the vest type item of clothing 2 comprises 2, 4, 6, 8, 12 or more conductive electrodes, preferentially 2, suitable for measuring electrical cardiac muscle activity 2.3. According to one embodiment, the means for measuring electrical cardiac muscle activity comprises a plurality of conductive electrodes not requiring gel for use. According to one embodiment, the means for measuring electrical cardiac muscle activity comprises a plurality of dry conductive electrodes. According to one embodiment, the means for measuring electrical cardiac muscle activity comprises a plurality of capacitive electrodes.

According to one embodiment, the vest type item of clothing 2 is suitable for being worn at the level of the cardiac muscle and comprises a plurality of conductive electrodes located on the vest type item of clothing 2 so as to be positioned at the level of the cardiac muscle when the vest type item of clothing 2 is worn by the subject. According to one embodiment, said conductive electrodes are conductive electrodes not requiring gel for use, preferentially said conductive electrodes are dry conductive electrodes.

According to one embodiment, the conductive electrodes suitable for measuring electrical cardiac muscle activity 2.3 are textile electrodes.

According to one embodiment, the vest type item of clothing 2 also comprises at least one ground 2.7 serving as a reference for the electrodes of the vest type item of clothing 2 so as to measure a difference in electric potential.

According to one embodiment, the vest type item of clothing 2 comprises a textile elastic strip 2.4, woven, knit or stitched in the vest type item of clothing 2 suitable for measuring respiratory muscle activity. According to one embodiment, said elastic strip 2.4 comprises a capacitive means for measuring the rib-cage spread, i.e. the change in rib-cage diameter. According to one embodiment, the vest type item of clothing 2 is suitable for being worn at the level of the rib-cage and comprises a textile elastic strip 2.4 located on the vest type item of clothing 2 so as to be positioned at the level of the rib-cage when the vest type item of clothing 2 is worn by the subject.

According to one embodiment, the vest type item of clothing 2 suitable for being worn on the subject's chest also comprises a means for measuring the blood oxygen concentration. According to one embodiment, the means for measuring the blood oxygen concentration is an oximeter located on the vest type item of clothing 2 so as to be positioned on the subject's chest, back or shoulder muscles when the vest type item of clothing 2 is worn by the subject.

According to one embodiment, the vest type item of clothing 2 suitable for being worn on the subject's chest also comprises a means for measuring electromagnetic radiation.

According to one embodiment, the vest type item of clothing 2 suitable for being worn on the subject's chest also comprises a means for measuring movement.

According to one embodiment, the vest type item of clothing 2 suitable for being worn on the subject's chest also comprises a means for measuring external electromagnetic interference.

According to one embodiment, the vest type item of clothing 2 suitable for being worn on the subject's chest also comprises a means for measuring the subject's body temperature.

According to one embodiment, the vest type item of clothing 2 also comprises a space suitable for receiving an electronic system 2.5 comprising the electrical signal acquisition means, signal processing means and transmission means. According to one embodiment the vest type item of clothing 2 also comprises a space suitable for receiving a battery 2.6 enabling the electrical power supply of the electronic system.

According to one embodiment, the signal processing means is situated in the cap type item of clothing 1 and/or in the vest type item of clothing 2. According to one embodiment, the signal processing means is situated at a distance from the cap type item of clothing 1 or the vest type item of clothing 2.

According to one embodiment, the signal processing means of the cap type item of clothing 1 and the signal processing means of the vest type item of clothing 2 are connected so as to be able to process equally the signals measured by the measuring means of the cap type item of clothing 1 and of the vest type item of clothing 2.

The present invention also relates to a device for monitoring the electro-physiological activity of a subject, particularly for diagnosing or monitoring epilepsy, comprising a cap type item of clothing 1 and a vest type item of clothing 2, the cap type item of clothing 1 and the vest type item of clothing 2 each comprising at least one means for measuring the electro-physiological activity of a subject, an acquisition means configured to acquire in synchronously the signals measured by the at least one means for measuring the subject's electro-physiological activity, a signal processing means and a means for transmitting the electro-physiological signals acquired by the acquisition means to said signal processing means. The signal processing means being configured to carry out the detections, identifications and processing operations mentioned above.

According to one embodiment, the means for measuring the electrical signals generated by the muscles of mastication is integrated in a textile portion, said textile portion including an elasticity coefficient suitable for adjusting the cap on a subject's head. According to one example, the means for measuring electrical signals are surface electrodes.

According to one embodiment, the textile portion surrounding each surface electrode is non-conductive and may form an electrical insulator.

According to one embodiment, the measuring means 1.2 in FIG. 1 are integrated in a textile element arranged on the lower part of the face, covering particularly a part of the surface of the cheeks and chin. The lower textile element including an attachment suitable for adjusting the left and right lateral parts on either side of the face. A benefit is that of offering a solution suitable for being adjusted to the heads of different subject profiles. An advantage is that of enhancing the acquisition of the electrical signals and therefore enhancing the processing carried out when these signals are compared or correlated or subtracted with respect to the signals acquired by the measuring means arranged on a subject's head.

According to one embodiment, the upper part of the cap covering the top of the head includes two layers. The layers are overlaid and form a space suitable for being closed. According to one example of an embodiment, the space is designed to receive the electrodes. A benefit is that of rendering the electrode wires invisible. According to one example, the cap includes at least one housing attached to the fabric including at least one battery powering the electrodes. An advantage is that of allowing the cap to be worn by a subject in order to render autonomous.

According to one embodiment, the textile element includes a means for holding said electrode in contact with a subject. According to a first case, the electrode is held by a strip including an attachment for holding a lateral strip on either side of a subject's face. According to a further case, the electrode is held by an attachment of the textile element to an upper part of a cap as shown in FIG. 1. In the latter case, the textile element is designed as a lower extension of the cap extending along the lower part of the face.

According to an example of an embodiment, the electrode of the means for measuring the electrical signals generated by the muscles of mastication is arranged in a strap of a cap including an upper part including electrodes for measuring electrical signals generated by brain activity. This strap may advantageously extend at the level of a subject's chin or neck.

The signals measured by the means for measuring electrical signals generated by the muscles of mastication are compared to the signals acquired by the means for measuring electrical brain activity signals. This comparison may previously be accompanied by signal processing steps such as the formatting thereof, amplification thereof, synchronisation thereof, denoising thereof or filtering thereof.

### BRIEF DESCRIPTION OF THE FIGURES

FIG. 1 is a diagram of the cap type item of clothing 1 according to one embodiment of the invention.

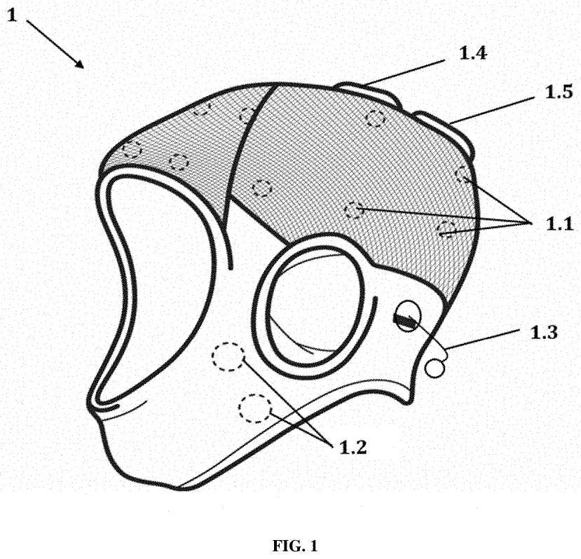

FIG. 2A is a front view diagram of the vest type item of clothing 2 according to one embodiment of the invention.

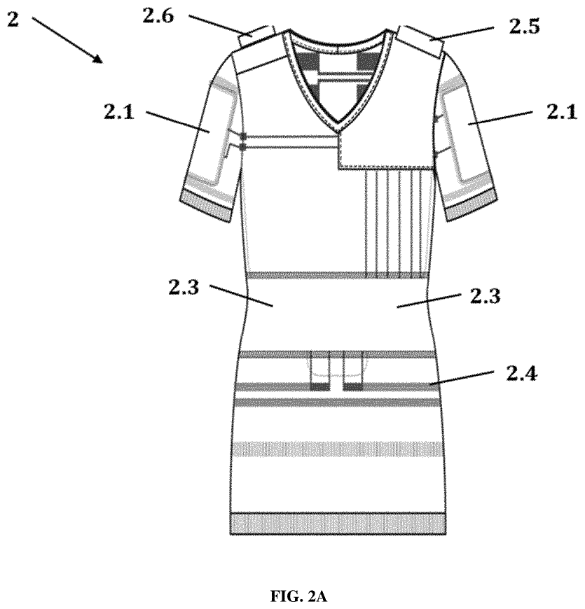

FIG. 2B is a rear view diagram of the vest type item of clothing 2 according to one embodiment of the invention.

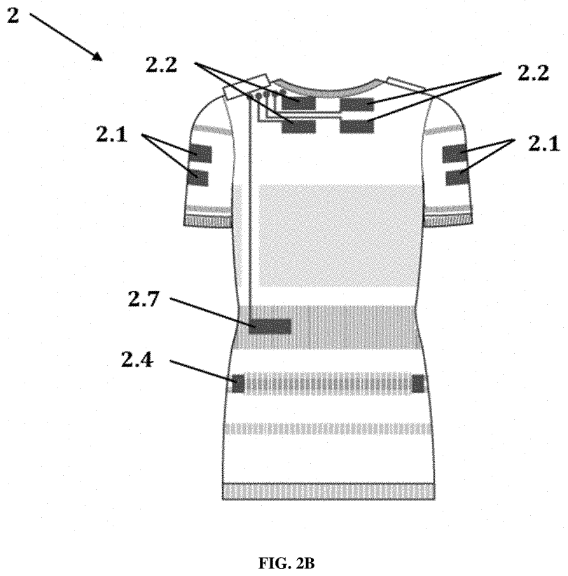

### REFERENCES

1 Cap type item of clothing;
1.1 Conductive electrode suitable for measuring electrical brain activity;
1.2 Conductive electrode suitable for measuring the electrical activity of the muscles of mastication;
1.3 Reference electrode;
1.4 Space suitable for receiving a battery;
1.5 Space suitable for receiving an electronic system;
2 Vest type item of clothing;
2.1 Conductive electrode suitable for measuring the electrical activity of the deltoid muscles;
2.2 Conductive electrode suitable for measuring the electrical activity of the trapezius muscles;
2.3 Conductive electrode suitable for measuring electrical cardiac muscle activity;
2.4 Strip for measuring respiratory muscle activity;
2.5 Space suitable for receiving an electronic system;
2.6 Space suitable for receiving a battery;
2.7 Ground of vest type item of clothing.

1-12. (canceled)

13. A device for monitoring the electro-physiological activity of a subject, particularly for diagnosing or monitoring epilepsy, comprising:
a measurement element for measuring electrical brain activity;
a measurement element for measuring the electrical activity of the muscles of mastication; the measurement element for measuring the electrical activity of the muscles of mastication including at least one surface electrode integrated in a textile element, said textile element including a holding element for holding said at least one electrode in contact with a portion of a subject's head;
an acquisition module configured to acquire synchronously the signals measured by the measurement element for measuring electrical brain activity and by the measurement element for measuring the electrical activity of the muscles of mastication;
a signal processing module; and
a transmission module for transmitting the electrical signals acquired by the acquisition module to said signal processing module;
wherein said signal processing module is configured to detect among the signals measured by the measurement element for measuring electrical brain activity the signals measured by the measurement element for measuring the electrical activity of the muscles of mastication.

14. The device according to claim 13, further comprising a measurement element for measuring the electrical activity of the deltoid muscles or the trapezius muscles, wherein the acquisition module is also configured to acquire synchronously the signals measured by the measurement element for measuring the electrical activity of the deltoid muscles or the trapezius muscles, and the signal processing module is also configured to detect among the signals measured by the measurement element for measuring the electrical activity of the deltoid muscles or the trapezius muscles signals correlated with predefined movements.

15. The device according to claim 13, further comprising a measurement element for measuring eye movement, wherein the acquisition module is also configured to acquire in a synchronised manner the signals measured by the measurement element for measuring eye movement, and the signal processing module is also configured to detect among the signals measured by the measurement element for measuring electrical brain activity the signals measured by the measurement element for measuring eye movement.

16. The device according to claim 13, further comprising a measurement element for measuring electrical cardiac muscle activity, wherein the acquisition module is also configured to acquire synchronously the signals measured by the measurement element for measuring electrical cardiac muscle activity, and the signal processing module is also configured to detect heartbeats among the signals measured by the measurement element for measuring electrical cardiac muscle activity.

17. The device according to claim 16, further comprising a measurement element for measuring respiratory muscle activity, wherein the acquisition module is also configured to acquire synchronously the signals measured by the measurement element for measuring respiratory muscle activity, and the signal processing module is also configured to detect among the signals measured by the measurement element for measuring electrical cardiac muscle activity parasitic signals correlated with the signals measured by the measurement element for measuring respiratory muscle activity.

18. The device according to claim 13, further comprising a measurement element for measuring external electromagnetic interference, wherein the acquisition module is also configured to acquire synchronously the signals measured by the measurement element for measuring external electromagnetic interference, and the signal processing module is also configured to detect among the signals measured by the measurement element for measuring electrical brain activity the signals measured by the measurement element for measuring external electromagnetic interference.

19. The device according to claim 13, further comprising a measurement element for measuring movement, wherein the acquisition module is also configured to acquire synchronously the signals measured by the measurement element for measuring movement, and the signal processing module is also configured to detect among the signals measured by the measurement element for measuring electrical cardiac muscle activity parasitic signals correlated with the signals measured by the measurement element for measuring movement.

20. The device according to claim 13, further comprising a measurement element for measuring the blood oxygen concentration and/or a measurement element for measuring electromagnetic radiation, notably visible electromagnetic radiation, and/or a measurement element for measuring body temperature.

21. The device according to claim 13, comprising a cap type item of clothing suitable for being worn on the subject's head and at the level of the subject's muscles of mastication; said cap type item of clothing comprising the measurement element for measuring electrical brain activity and the measurement element for measuring the electrical activity of the muscles of mastication; said measurement element for measuring electrical brain activity comprising a plurality of conductive electrodes, and said measurement element for measuring the electrical activity of the muscles of mastication comprising at least one conductive electrode.

22. The device according to claim 14, comprising a vest type item of clothing suitable for being worn on the subject's chest; said vest type item of clothing comprising the measurement element for measuring the electrical activity of the deltoid muscles or trapezius muscles, the measurement element for measuring electrical cardiac muscle activity and/or the measurement element for measuring respiratory muscle activity; said measurement element for measuring the electrical activity of the deltoid muscles or trapezius muscles comprising a plurality of conductive electrodes; said measurement element for measuring cardiac muscle activity comprising at least one conductive electrode; and said measurement element for measuring respiratory muscle activity comprising a textile elastic strip.

23. The device according to claim 22, wherein the vest type item of clothing comprises a capacitive measurement element for measuring the rib-cage spread.

24. The device according to claim 21, wherein the cap type item of clothing is a textile item of clothing, and the conductive electrodes comprise textile electrodes woven, knit, stitched, deposited or printed in or on said items of textile clothing.

25. The device according to claim 22, wherein the vest type item of clothing is a textile item of clothing, and the conductive electrodes comprise textile electrodes woven, knit, stitched, deposited or printed in or on said items of textile clothing.

26. The device according to claim 13, wherein said signal processing module comprises a memory unit, said memory unit comprising neurological markers; and the signal processing module is configured to identify a neurological disorder among the signals measured by the measurement element for measuring electrical brain activity based on the neurological markers.
